(19) United States
(12) Patent Application Publication
(10) Pub. No.: US 2003/0096001 A1
(43) Pub. Date: May 22, 2003

(54) Encapsulation products and method of controlled release of fluoxetine or mesalamine

(76) Inventors: S. Rao Cherukuri, Frederick, MD (US); Vittorino Ravelli, Milano (IT)

Correspondence Address: Gary M. Nath, NATH & ASSOCIATES PLLC, 1030 15th Street, N.W. - 6th Floor, Washington, DC, 20005, US

(21) Appl. No.: 10/209,659

(22) Filed: Aug. 1, 2002

Related U.S. Application Data
(63) Continuation-in-part of application No. 09/982,092, filed on Oct. 19, 2001.
Application No. 09/982,092 is a continuation-in-part of application No. 09/587,971, filed on Jun. 6, 2000.
(60) Provisional application No. 60/344,331, filed on Jan. 4, 2002.

(51) Int. Cl. A61K 9/48; A61K 9/62

(52) U.S. Cl. 424/452; 424/461

(57) ABSTRACT

A novel extended or controlled release encapsulated product is provided and includes: a pharmaceutically effective amount of fluoxetine HCl or mesalamine; at least one erodible polymer; and at least one lubricating material; wherein the encapsulated product is in the form of a caplet having a diameter of from about 1 millimeter to about 7 millimeters and a length from about 1 millimeter to about 7 millimeters. A pulsating release product for extended release of fluoxetine is also disclosed. A method for preparing the encapsulated product is also provided.

### BACKGROUND OF THE INVENTION

1. Field of the Invention

The present invention relates to an encapsulation process, and in particular, an alternate encapsulation process for concentrating additives using compression. Also, the present inventive subject matter relates to encapsulation products that provide controlled or extended release of the anti-depressant drug fluoxetine or the anti-inflammatory (gastrointestinal) drug mesalamine, thereby providing a convenient single dose for maintenance therapy without providing an increase in undesirable side effects.

2. Description of the Prior Art

Various types of chewable articles are known in commerce. These articles include food items such as food items, confectionery items and chewing gum. The chewable articles often include various types of active agents or ingredients within the chewable articles. Examples of such active ingredients include flavors, sweeteners, colors, medicaments, pharmaceuticals, vitamins, minerals, and other effervescent agents.

It has been known in the art of food stuff, confectionery and chewing gum preparation to provide protection to the active ingredients by the use of protection systems, including providing a protective coating around the active ingredient or encapsulating the active ingredient. Such protective systems have been employed for various reasons, such as for protection of the active ingredient, both while on the shelf and during use, and for prolonged release in the oral cavity.

It is known in the art to protect active ingredients by encapsulating the active ingredient prior to introducing the ingredient into a final product. Some of the major classifications of encapsulation technology include liquid suspending media (water-in-oil emulsions and oil-in-water emulsions), interfacial and in situ polymerization, solvent evaporation from emulsions, desolvation, complex coacervation, polymer and polymer incompatibality, gelation, and pressure extrusion. One of skill in the art will be familiar with each of these classifications.

Schobel, U.S. Pat. No. 4,568,560, discloses encapsulated fragrances and flavors for use in denture cleanser compositions. Schobel discloses encapsulating a solid particulate flavoring agent or fragrance with a film of an acrylic polymer and ethylcellulose. The encapsulation is accomplished utilizing a fluidized bed of the flavoring agent or fragrance.

Yang, U.S. Pat. No. 4,740,376, discloses encapsulating an active ingredient in a solvent free encapsulation composition which includes a blend of a high molecular weight polyvinyl acetate and a hydrophilic plasticizer. The active ingredient is protected from deterioration due to moisture and is provided with controlled release for use in a product to be ingested by a mammal.

Cherukuri et al., U.S. Pat. No. 4,981,698, discloses a delivery system for sweeteners that comprises a first high intensity sweetener encapsulated in a first core coating, and a second outer hydrophilic coating containing up to the solubility limit of the second coating of a second sweetener. The delivery system offers enhanced up front sweetness intensity in combination with prolonged sweetness duration, and improved protection and stability of the sweetener.

Cherukuri et al., U.S. Pat. No. 5,004,595, discloses a free-flowing particulate delivery system for providing enhanced flavor and sweetness to comestible products. The delivery system includes an encapsulating matrix that protects flavor in a core.

Cherukuri et al., U.S. Pat. No. 5,266,335, discloses microencapsulated flavoring agents and methods for preparing the same. The microencapsule comprises a flavoring agent and a resin in the core, and a coating layer over the core. The core is encapsulated by emulsion of a flavoring agent and a resin with a coating layer prepared by complex coacervation of a mixture of two or more colloidal materials.

Kehoe, U.S. Pat. No. 4,975,270, discloses elastomer encased active ingredients. The active ingredients are physically encased in non-porous, chewable particles of elastomer. The particles are then incorporated into articles of commerce.

As is seen above, historically, the most convenient and commonly employed route of drug delivery has been by oral ingestion. The original controlled release of pharmaceuticals was through coated pills which dates back over 1000 years. Coating technology advanced in the mid- to late 1800s with the discovery of gelatin and sugar coatings. A major development in coating technology was the concept of coating drug-containing beads with combinations of fats and waxes. Since the mid-1900s, hundreds of publications and nearly a thousand patents have appeared on various oral delivery approaches encompassing delayed, prolonged, suspended and most recently, controlled release of active ingredients.

In the mid- to late 1960s, the term controlled drug delivery came into being to describe new concepts of dosage form design. These concepts usually involved controlling drug dissolution but also had additional objectives. The primary objectives of a controlled-release system have been to enhance safety and extend duration of action. Today, controlled-release systems are designed in order to produce more reliable absorption and to improve bioavailability and efficiency of delivery.

The overwhelming majority of controlled release systems rely on dissolution, diffusion, or a combination of dissolution and diffusion to generate slow release of a drug. Ueda et al., U.S. Pat. No. 4,874,549, disclose a time-controlled system in which a drug is diffused into a patient after the explosion of a membrane at a given period of time after ingestion. The system is comprised of a preparation in the form of a bead or granule which makes up a core, a drug, a swelling agent and an outer membrane made up of a water-insoluble coating material.

Chen, U.S. Pat. No. 5,508,040, discloses a multiparticulate pulsatile drug delivery system. The system is comprised of a large number of pellets containing a drug and a water soluble osmotic agent. The pellets are an agglomerate of sugar seeds with the drugs spray-coated thereon.

Philippon et al., U.S. Pat. No. 5,229,135, disclose a sustained release diltiazem formulation. The formulation is ingested orally and, like Chen above, the core is a central sugar sphere with a plurality of coatings in which the drug is adhered to the sphere.

Chen, U.S. Pat. No. 5,567,441, also discloses a diltiazem controlled release formulation to be ingested orally. And like Chen and Philippon above, the core is a non-pareil or sugar bead on which the drug is applied via a coating.

However, there are a number of perceived disadvantages with regards to controlled release of drugs into a system. The disadvantages include a longer time to achieve therapeutic blood concentrations, possible increased variation in bioavailability after oral administration, enhanced first-pass effect, dose dumping, sustained concentration in overdose cases, lack of dosage flexibility and, often, greater expense. It should be pointed out that there are a number of constraints on the design of oral controlled drug delivery systems: dose size, drug molecular size, charge and pKa, aqueous solubility, partition coefficient, stability, absorption, metabolism, half-life, margin of safety, toxicity, and clinical response.

In addition, there are a number of disadvantages when using the traditional encapsulation processes to encapsulate active ingredients, including pharmaceuticals and nutriceuticals. The disadvantages include the need for heat and moisture in order to properly form the encapsulated final product. Also, most encapsulation methods are complex and consume large amounts of time in order to obtain the final encapsulated product. Further, current encapsulated ingredients vary in size from nanometers to about 400 microns, and the active ingredients are not uniformly distributed throughout the encapsulated product.

Therefore, there remains a need for an alternate encapsulation method for providing a controlled or extended release product with high levels of active ingredients and in which water is not needed during the encapsulation process, nor is heat an essential feature of the encapsulation process. There also remains a need for an alternate encapsulation method which produces capsules with uniform active ingredient content throughout the product, and that can withstand mechanical pressure both in the processing of the capsule and in the chewing of the product in the mouth so that the active ingredients are released in the stomach of the consumer. Further, there remains a need for a simple encapsulated product that provides good controlled and extended release characteristics for pharmaceuticals.

Fluoxetine (N-methyl-3-(p-trifluoromethylphenoxy)-3-phenyl-propylamine) is an antidepressant drug which is disclosed, for example, in U.S. Pat. Nos. 4,314,081 and 4,626,549. The action of fluoxetine is based on its capacity to selectively inhibit the uptake of serotonin by the neurons in the central nervous system. Fluoxetine is indicated in the U.S. and many other countries for the treatment of depression, obsessive-compulsive disorder, and bulimia.

In the U.S., the currently available pharmaceutical forms for fluoxetine, in the form of the hydrochloride salt, include capsules and a solution. A tableted formulation for compounds of the fluoxetine type is also contemplated in U.S. Pat. No. 4,314,081 (column 16, lines 52-55). More recently, a dispersible tablet has been disclosed (see EPO Patent application publication 693,281). A sustained release formulation of fluoxetine is claimed in U.S. Pat. No. 4,847,092. Tablets of serotonin uptake inhibitors which are coated to delay absorption and disintegration to “provide a sustained action over a longer period” are generally contemplated in U.S. Pat. No. 4,444,778 (column 6, line 10 et seq.). Formulations of R-fluoxetine are generally contemplated in WO 92/13452 (controlled release and sustained release—page 19) and U.S. Pat. No. 5,356,934 (column 4). Similar teaching for S-fluoxetine are found in U.S. Pat. No. 5,104,899.

Enteric pharmaceutical formulations are manufactured in such a way that the product passes unchanged through the stomach of the patient, and dissolves and releases the active ingredient quickly when it leaves the stomach and enters the small intestine. Such formulations have long been used, and conventionally are in tablet or pellet form, where the active ingredient is in the inner part of the tablet or pellet and is enclosed in a film or envelope, the “enteric coating”, which is insoluble in acid environments, such as the stomach, but is soluble in near-neutral environments such as the small intestine.

Certain difficulties arose in preparing conventional enteric formulations of fluoxetine. In particular, fluoxetine was found to react with many enteric coatings to form a slowly—or even insoluble coating. Similar reactions with enteric coatings have been observed with other drugs—duloxetine, nortriptyline, desipramine, sertraline and paroxetine. It has been observed that, because of fluoxetine's long half life, dosing regimens other than daily dosing are effective, especially for maintenance dosing. For example, Burke, et al., Psychopharmacol. Bull., 31(3), 524 (1995) reported that 60 mg of fluoxetine hydrochloride given once per week was as effective as 20 mg per day during maintenance therapy (i.e., after eight weeks of daily dosing). Montgomery, et al., Eur. Arch. Psychiatry Clin. Neuroscience, 244(4), 211 (1994) reported that 120 mg of fluoxetine dosed biweekly was ineffective for treating recurrent brief depression. Twenty milligrams per week of fluoxetine were advocated by Benazzi, et al., Pharmacopsychiatry, 27(6), 246 (1994), for reducing sexual dysfunction side effects. While the above studies employed single or multiple 20 mg capsules to provide the indicated therapy, 60 mg capsules of fluoxetine hydrochloride are available in, e.g., South Africa for treating bulimia.

Because of fluoxetine's long half life, there has not been any perceived need to actually prepare a fluoxetine formulation providing a longer payout. While these higher doses of fluoxetine have been shown to be efficacious, there can be associated side effects, such as nausea, presumably due to local irritation or the increased plasma levels shortly after dosing. Therefore, it has now been appreciated that a formulation having higher doses of fluoxetine (e.g., 60-120 mg) which blunts the initial release of fluoxetine will have clinical advantages, i.e., not only will such formulations provide convenient and effective one per week dosing, but will have an advantage of less side effects.

It is therefore desirable to have a formulation that could be used to provide a convenient single dose, i.e. 90 mg once weekly for maintenance therapy suggested by the above articles without providing an increase in undesirable side effects.

U.S. Pat. No. 5,910,319 disclose an enteric formulation of the anti-depressant drug fluoxetine, in the form of enteric pellets comprising a) an inert core consisting of fluoxetine and one or more pharmaceutically acceptable excipients that has been applied to the core prepared from starch and sucrose, for example; b) an optional separating layer; c) an enteric layer comprising hydroxypropyl-methyl cellulose acetate succinate (HPMCAS) and a pharmaceutically acceptable excipient; and d) an optional finishing layer. A “critical element” of the '319 patent is the teaching that reducing sugars such as lactose are to be avoided because fluoxetine interacts with lactose both at room temperature and under accelerated stability conditions (heat).

Moreover, attention must be given to the particle size of fluoxetine. The compound can precipitate in needle-like crystals which can be quite large. Coating cores with fluoxetine in the large needle-like form can be difficult, and it is advisable to mill or otherwise reduce the particle size of the fluoxetine to less than about 50 μm before using it in the product and process disclosed by the '319 patent. Coating may be performed using “powder coating”, “fluidized bed” or “spray slurry” processes.

The present invention was created through efforts to solve the above and other problems, and provides a superior enteric formulation of fluoxetine.

### BRIEF SUMMARY OF THE INVENTION

Applicant has unexpectedly produced an encapsulated product having extended or controlled release of fluoxetine, comprising:

a) a pharmaceutically effective amount of fluoxetine HCl;

b) at least one pharmaceutically acceptable excipient;

c) at least one erodible polymer; and

d) at least one lubricating material; and

e) wherein said product is in the form of a caplet having a diameter from about 1 millimeter to about 7 millimeters and a length from about 1 millimeter to about 7 millimeters.

In a preferred embodiment of the present inventive subject matter, the erodible polymer is a water soluble polymer.

In another preferred embodiment, the erodible polymer is a water insoluble polymer.

A further preferred embodiment is drawn to a pulsating release encapsulated product, comprising:

a) a pharmaceutically effective amount of fluoxetine HCl;

b) at least one pharmaceutically acceptable excipient;

c) at least two erodible polymers, each of said erodible polymers having a different rate of dissolution or dissolving at a different pH; and

d) at least one lubricating material; and

e) wherein said product is in the form of a caplet having a diameter from about 1 millimeter to about 7 millimeters and a length from about 1 millimeter to about 7 millimeters.

A still further preferred embodiment is drawn to a pulsating release product, comprising a capsule having a plurality of caplets, said caplets comprising:

a) a pharmaceutically effective amount of fluoxetine HCl;

b) at least one pharmaceutically acceptable excipient;

c) at least one erodible polymer;

d) at least one lubricating material; and

e) wherein said caplet has a diameter from about 1 millimeter to about 7 millimeters and a length from about 1 millimeter to about 7 millimeters; and

wherein at least one of said plurality of caplets is prepared from an erodible polymer having a first dissolution rate, and at least another of said plurality of caplets is prepared from another erodible polymer having a second dissolution rate, and said first dissolution rate is not equal to said second dissolution rate.

An advantage of the method of the inventive subject matter is that no heat nor moisture is required for forming the encapsulated product. High levels of fluoxetine HCl or mesalamine are obtainable in the products of the inventive subject matter, even though heat or moisture is not required for forming the encapsulated product. In addition, the encapsulated product of the present inventive subject matter has a uniform active ingredient content and may be strong enough to withstand mechanical pressure both in the processing of the product, and in the chewing of the product in the mouth so that the fluoxetine Hcl or mesalaime is released in the stomach.

Yet another advantage of the product and method of the inventive subject matter is that they may be used with reducing sugars such as lactose which is contrary to the teaching of the fluoxetine formulation of the '319 patent.

Still another advantage is that the method and product does not require the additional processes required to coat fluoxetine on the inert core such as “powder coating”, “fluidized bed” or “spray slurry” processes.

### BRIEF DESCRIPTION OF THE DRAWINGS

FIG. 1 is a schematic diagram illustrating the structure of the caplet of the encapsulation product.

FIG. 2 is a comparative dissolution profile of the 90-mg fluoxetine encapsulation products of the present inventive subject matter for Examples 1-3, 6 and 8.

FIG. 3 is a comparative dissolution profile of the 90-mg fluoxetine encapsulation products of the present inventive subject matter for Example 5.

### DETAILED DESCRIPTION OF THE INVENTION

The encapsulated product of the present invention is a caplet containing a surprisingly high amount of fluoxetine or mesalamine providing excellent controlled or extended release properties. Applicants have unexpectedly determined that fluoxetine or mesalamine can be compressed with high load into a small encapsulated product.

The controlled release products of the present inventive subject matter are designed to produce a sustained concentration of fluoxetine or mesalamine in the blood. The advantages of the controlled or extended release products of the present inventive subject matter include reduced toxicity and sustained efficacy of the fluoxetine; decreased frequency of dosing, resulting in improved patient compliance, reduced patient care; and possibly reduced amount of drug used.

In a preferred embodiment of the present invention, the controlled or extended release encapsulated product of the present inventive subject matter is a caplet shaped like a capsule and having a diameter from about 1 millimeter to about 7 millimeters and a length from about 1 millimeter to about 7 millimeters. Preferably, the diameter of the encapsulated product is about 3 millimeters and the length is about 3 millimeters. The caplets may be coated with a thin surface film to protect the product from moisture or water absorption, from flavor release in the final product system, and from heat and rupture during processing and chewing.

As used herein, the expression “mammal” includes without limitation any mammalian subject, such as mice, rats, guinea pigs, cats, dogs, human beings, cows, horses, sheep or other livestock.

As used herein, the term “fluoxetine” means Fluoxetine (N-methyl-3-(p-trifluoromethylphenoxy)-3-phenylpropylamine), solvates of fluoxetine or its salts as well as the free base, salts, and/or solvates of the individual isomers of fluoxetine. A preferred salt of fluoxetine is fluoxetine hydrogen chloride or fluoxetine HCl. Throughout this description, unless specified otherwise, the term “fluoxetine” contemplates all such forms.

As used herein, “mesalamine” means mesalamine (5-Amino-2-hydroxybenzoic acid), solvates of mesalamine or its salts as well as the free base, salts and/or solvates of the individual isomers of mesalamine. Throughout this description, unless specified otherwise, the term “mesalamine” contemplates all such forms.

As used herein, “controlled release” or “extended release” relates to the release rates of the fluoxetine from the encapsulated product into the mammal. The terms refer to the release of the drug over a period of time, for example from one hour to twenty-four hours.

Currently, controlled release formulations are generally created through different technological approaches which include:

Drug loading on spherical pellets, can be accomplished through either solvent based or aqueous coatings.

Monolithic (tablets) dosage forms based on hydrophyllic swelling polymers in which the drug is dispersed and then released through hydrated swollen matrix.

Erodible matrixes, either multi-particulate or monolithic, that release the included active substance, generally poorly water soluble, by controlled erosion of the system.

Osmotic systems in monolithic, tablets, form that release the drug, soluble in digestive fluids, through a calibrated hole in the osmotic membrane surrounding the tablet.

In a preferred embodiment, the fluoxetine or mesalamine is incorporated into an erodible polymer matrix. A general method for preparing a controlled-release encapsulated product encompasses the following steps. First, the fluoxetine or mesalamine is mixed with a suitable erodible polymer. The fluoxetine or mesalamine may be present in amounts from 0.001 to 70.0% by weight of the final encapsulated product. The erodible polymer may be present from 10.0 to 70.0% by weight of the final encapsulated product.

The present inventive subject matter contemplates that the erodible polymer may be either water soluble or water insoluble. Water soluble polymers useful in the present inventive subject matter include, without limitation, sodium carboxymethyl cellulose, hydroxyethyl cellulose, hydroxypropyl cellulose, propylene glycol alginate, sodium alginate, carboxymethyl cellulose and mixtures thereof.

Likewise, water insoluble polymers that are useful in the present inventive subject matter include, without limitation, cellulose acetate, ethyl cellulose, cellulose acetate methyl carbamate, methylcarbamate, polydiethylaminomethylstyrene, ethyl cellulose, cellulose acetate, cellulose diacetate, cellulose triacetate, cellulose alkanylate, monoalkenytes, dialkenytes, trialkenytes, mono-, di- and tri-arolyates, cellulose trivalerate, cellulose trioctanoate, cellulose tripionate, celluslose diesters, cellulose disuccinate, cellulose acetate valerate, cellulose acetaldehyde, dimethylcellulose acetate, cellulose dimethylaminoacetate, semipermeable sulfonated polystyrenes, semipermeable styrenes, hydroxypropylmethyl cellulose and mixtures thereof.

The fluoxetine or mesalamine/polymer mixture is then granulated using a suitable binder. The binder is generally present in amounts of 1.0 to 10.0% by weight of the final encapsulated product. Binders suitable for use in the present inventive subject matter include, without limitation, plasdone K-29/32, povidone K30, carboxymethylcellulose sodium, ethylcellulose, methylcellulose, alginic acid and mixtures thereof.

After the active ingredient/polymer mixture is granulated, the mixture is passed through a mesh, preferably a mesh no. 30, and allowed to air dry. After air-drying, the mixture may be passed through another mesh, preferably a no. 20 mesh.

After passing through the second mesh, the granulated mixture is lubricated with a lubricant and a binder and compressed into capsules with the sizes listed above. The lubricant or lubricating material forms a film around the granules and helps the material flow, compress and eject from the tableting machine. The lubricant or lubricating material may be present in levels up to 5% by weight of the final composition. Examples of usable lubricating materials include, without limitation, fats, emulsifiers, waxes, magnesium stearate, calcium stearate, talc, starches, silicon dioxide, and mixtures thereof. Among the fats, or fatty materials, useful herein include, without limitation, water-insoluble, inert hydrocarbon fats or oils, or their derivatives and mixtures thereof. Such fats or fatty materials include, for example and without limitation, cocoa butter, hydrogenated vegetable tallow, hydrogenated vegetable oils, and derivative mixtures thereof.

Among the emulsifiers useful herein include, without limitation, alkyl aryl sulfonates, alkyl sulfates, sulfonated amides and amines, sulfated and sulfonated esters and ethers, alkyl sulfonates, polyethoxylated esters, mono- and diglycerides, diactyl tartaric esters of monoglycerides, polyglycerol esters, sorbitan esters and ethoxylates, lactylated esters, propylene glycol esters, sucrose esters and mixtures thereof. Among the waxes useful herein include, without limitation, amorphous waxes, anionic emulsifying waxes, bleached waxes, caranda waxes, cetyl esters, cationic emulsifying waxes, microcrystalline waxes, paraffins, refined waxes and mixtures thereof.

The use of particular fats, emulsifiers or waxes may allow the encapsulated product of the present inventive subject matter to aid in providing controlled or sustained release of the fluoxetine or mesalamine. The controlled release occurs due to the entrapment of the fluoxetine or mesalamine in the particular fat, emulsifier or wax.

It is possible to provide a coating on the encapsulated product. The coating provides protection of the active ingredient from moisture or water absorption. The coating may also allow the release of the active ingredient in the stomach of the individual, and not in the mouth thereof. It is preferred that the water insoluble polymer cellulose acetate phthalate be used as the coating.

The aforementioned caplet thus contains a pharmaceutically effective amount of fluoxetine or mesalamine and at least one pharmaceutically acceptable excipient, preferably lactose. At least one erodible polymer is also contained in the caplet, preferably hydroxypropylmethyl cellulose with at least one lubricating material, as listed above. The caplet has a diameter of about 1 millimeter to about 7 millimeters and a length of about 1 mm to about 7 mm, as previously discussed.

It should be noted that the structural arrangement of the caplet containing the fluoxetine and the lactose and other excipients, disperses the lactose and reduces its interaction with the fluoxetine greatly reducing the incompatibility problem noted in the '319 patent.

Moreover, the sustained drug release accomplished by the embodiments of the inventive subject matter results in the release of the fluoxetine or mesalamine at the colon below the small intestine. Conversely, the formulation of the '319 patent results in release at the duodenum and the small intestine.

In one aspect of the inventive subject matter the encapsulated product is coated with a polymeric coating to form an extended release formulation. In this aspect the extended release formulations are where the mechanism of release is driven, predominantly, by the osmotic pressure.

In the present procedure, the encapsulated products are formulated with osmotic ingredients and coated with semi-permeable film forming polymers to achieve zero-order release. The advantages of this formulation include the combination of the mechanism of the control of the release based on the osmotic pressure (finely tuned and independent from the motility, pH, composition of the digestive fluids and food) with the concept of multiple units (improved inter and intra-subjects variability of absorption).

Any inorganic salt may be used which is highly dissociated in aqueous media in the range of pH from 1 to 7 and suitable to be included in pharmaceutical preparations for oral administration.

Semi-permeable film forming polymers can be high molecular weight derivatives of cellulose which are insoluble in water as ethylcellulose with a degree of ethylation between 43% and 50%, cellulose acetate with 30%-45% of acetyl value, polyvinylacetate, ammonium methacrylate co-polymers.

Suitable plasticizers can be added in the range of 5% to 35%. The film thickness may vary from 20 μm to 1000 μm to achieve the desired extended release profile. The size of encapsulated products may vary between 2 mm to 3 mm of diameter and height. Depending on the composition of the core and on the type and thickness of the film different zero order kinetics can be achieved.

In another aspect of the inventive subject matter, the encapsulated product is coated with a polymeric coating to form a delayed release formulation. In this aspect, the delayed release, monolithic, oral dosage form is based on osmotic pressure and then a crown coating in the coating polymer formed in situ when the dose is ingested.

Oral dosage forms have heretofore been generally based on osmotic pressure by preparing a core, usually in the form of a tablet, containing the active substance dispersed in a combination of ingredients able to generate an osmotic pressure, when contained by an osmotic membrane. The release from such systems is obtained with a calibrated hole in the membrane which regulates, together with the level of osmotic pressure generated by the intrinsic characteristics of the tablet core, the rate of release.

In the present invention, tablets with swelling polymers, osmotic ingredients and fluoxetine, with a cone protuberance on one side are then coated with semi-permeable film-forming polymer. The thickness of the film is lower on the cone. By swelling, the film brakes on the cone because the lower thickness and the content of the vesicle (the coated tablet) is released. For given dimensions and composition of the tablet, the time to break is regulated by the thickness of the film and the height of the cone. This system allows to match the result without the need of holing the osmotic film with a laser beam, risk of irradiated polymers with free radicals, or by using a micro drill, a costly process.

Any inorganic salt may be used which is highly dissociated in aqueous media in the range of pH from 1 to 7 and suitable to be included in pharmaceutical preparations for oral administration.

Swelling polymers can be polycarbophyls and hydroxypropylmethylcellulose of different viscosity such as from 4,000 to 100,000 cps.

The diameter of the tablets can vary from 5 mm to 10 mm.

The thickness can vary from 3 mm to 5 mm.

The height of the cone can vary from 0.5 mm to 1.0 mm.

The shape of the protuberance is conical with the diameter of the base that can vary from 0.8 to 1.3 mm.

Semi-permeable film forming polymers can be high molecular weight derivatives of cellulose which are insoluble in water as ethylcellulose with a degree to ethylation between 43% and 50%, cellulose acetate with 30%-45% of acetyl value, polyvinylacetate, ammonium methacrylate co-polymers.

Suitable plasticizers can be added in the range of 5% to 35%. The film thickness may vary from 20 μm to 150 μm.

This formulation can be applied to any modified release applications including OTC & Rx Pharmaceuticals, and Nutritional applications.

In a preferred embodiment of the present inventive subject matter, the controlled release of the encapsulated product provide “pulses” or “pulsating release” of the fluoxetine or mesalamine. By “pulses” or “pulsating release”, Applicants mean that the fluoxetine or mesalamine is released at different time intervals while in the body of the mammal. By “pulsating,” Applicants also mean that the release of the drug may be continuous, discontinuous, extended or sustained. Preferably, the “pulsating” aspect of the release of the fluoxetine means the discontinuous release of the drug.

Fluoxetine needs frequent administration of burst doses in order to achieve optimal effect. The most common way of achieving pulsating release has been to coat the drug with slowly dissolving polymeric membranes or with protective polymers that dissolve selectively at pH's corresponding to specific regions of the gastrointestinal tract. Once the polymeric membrane has dissolved, all of the fluoxetine inside the membrane is immediately available for dissolution and absorption. Thus, the fluoxetine release can be controlled by adjusting the thickness and dissolution rate of the polymeric membrane surrounding the drug. If only a few different thicknesses of membrane are used, the fluoxetine will be released at different, predetermined times, or “pulses.” The present inventive subject contemplates coating the encapsulated products with such polymeric membranes, as is discussed above.

In addition, however, the present inventive subject matter provides pulsating delivery of the fluoxetine by taking advantage of the characteristics of the different erodible polymers used in the encapsulated products. In a preferred embodiment of the present inventive subject matter, the encapsulated product is prepared with at least two erodible polymers, each having a different rate of dissolution in the body of the mammal in which the encapsulated product is introduced.

For example, the encapsulated product may be made with two or more erodible polymers, at least one that erodes quickly in the body to provide immediate dissolution of the fluoxetine and at least another that does not erode as quickly, thus delaying release of the fluoxetine until a desired time.

These characteristics of the present inventive subject matter with respect to the release of fluoxetine also apply to caplets in which mesalamine is incorporated as the active ingredient in place of the fluoxetine. It is contemplated that the present inventive subject matter equally covers fluoxetine and mesalamine.

As is stated above, an important aspect of this embodiment of the present inventive subject matter is the incorporation of at least two erodible polymers having different rates of dissolution. The present inventive subject matter contemplates the use of both water soluble polymers and water insoluble polymers for this preferred embodiment. Examples of water soluble and water insoluble polymers are listed above. Depending on the desired characteristics of the encapsulated product and release profile of the fluoxetine or mesalamine used, the erodible polymers used to achieve the pulsating release of the active ingredient may be water soluble, water insoluble, or a mixture thereof. One of ordinary skill in the art will be able to easily determine which polymers are suitable to achieve the desired pulsating release of the active ingredients based on the dissolution rates of the various erodible polymers.

In a further embodiment of the present inventive subject matter, the pulsating effect is achieved by incorporating into a standard capsule encapsulated products prepared from different erodible polymers. In this embodiment, the fluoxetine or mesalamine is incorporated into multiple encapsulated products using two or more different erodible polymers, with each encapsulated product being prepared with a different erodible polymer. Then, the different encapsulated products are included in a standard capsule which is taken by the mammal. The encapsulated products will erode at different rates based upon the erodible polymers with which the products were made, providing a pulsating release profile of the active ingredients. Again one of ordinary skill in the art will be able to easily determine which polymers are suitable to achieve the desired pulsating release of the fluoxetine or mesalamine based on the dissolution rates of the various erodible polymers.

The structural arrangement of the caplet containing the aforementioned ingredients and its attendant advantages are the same as the single extended release encapsulated product, except that multiple erodible polymers and caplets are used.

While the above delivery of controlled release encapsulated products is by oral ingestion of the encapsulated products, the present inventive subject matter also contemplates site-specific delivery of the active ingredient by different modes of introduction of the drug into the body. The different modes include, for example, introduction of the encapsulated products rectally, which will allow introduction of the products directly into the large bowel of the mammal. In this way, the encapsulated products will act much like a suppository, providing controlled release of the fluoxetine while at the same time by-passing the oral route of delivery.

In the above non-oral delivery embodiments, the controlled release encapsulated products may have the same characteristics as described above for oral delivery of active ingredients. That is, the controlled release may be zero order, or may provide a pulsating effect, as is defined above. Further, the pulsating effect may be the result of two or more different erodible polymers being incorporated into the same inventive encapsulated product, or the pulsating effect may be due to the presence of multiple encapsulated products having been made from different erodible polymers having different rates of dissolution.

The above non-oral delivery routes of fluoxetine or mesalamine are meant as non-limiting examples only, and it should be recognized that other non-oral delivery routes are also within the contemplation of the present inventive subject matter.

The amount of fluoxetine or mesalamine present in the inventive compositions may vary but generally will be present in an amount of about 0.001% to 70% by weight of the composition. In particular, fluoxetine HCl can be formulated with the present invention in doses ranging from about 10 to 90 mg. Likewise, mesalamine can be formulated with the present invention in doses also ranging from 10 to 250 mg. One of ordinary skill in the art will be able to determine the proper dosage for the remaining disclosed drugs.

In a further embodiment of the present invention, the encapsulated product includes the incorporation of flavors. The flavoring agents which may be used include those flavors known to the skilled artisan, such as natural and artificial flavors. These flavorings may be chosen from synthetic flavor oils and flavoring aromatics and/or oils, oleoresins and extracts derived from plants, leaves, flowers, fruits, and so forth, and combinations thereof. Nonlimiting representative flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including, without limitation, lemon, orange, lime, grapefruit, and fruit essences including apple, pear, peach, grape, strawberry, raspberry, cherry, plum, pineapple, apricot and so forth. These flavoring agents may be used in liquid or solid form and may be used individually or in admixture. Commonly used flavors include mints such as peppermint, menthol, artificial vanilla, cinnamon derivatives, and various fruit flavors, whether employed individually or in admixture.

Other useful flavorings include aldehydes and esters such as cinnamyl acetate, cinnamaldehyde, citral diethylacetal, dihydrocarvyl acetate, eugenyl formate, p-methylamisol, and so forth may be used.

If the flavor to be added is liquid, then the liquid flavor is first absorbed onto a solid absorbent. Examples of absorbents on which the liquid may be absorbed include, without limitation, silica gel particles, starches, carbohydrates such as sugars and polyhydroxyalcohols, celluloses, calcium salts such as calcium phosphate, calcium carbonate, and calcium sulfonate, and other absorbing agents in free-flowing powder form. The amount of liquid flavor added depends on the final concentration desired. Generally, though, the liquid flavor will be present in quantities from about 0.1% to 70% by weight of the resultant flavor/absorbent mixture.

The flavor/absorbent mixture is then mixed with a the erodible polymer above.

Furthermore, other additives such as colors, may also be added to this mixture to form the final mixture. The final mixture is then formed into the encapsulated product of the present invention by using a tableting machine. The stations of the tableting machine are set to the desired caplet size, which is from about 1 millimeter to about 7 millimeters diameter and length for the encapsulated.

The use of flavor with fluoxetine or mesalamine in the encapsulated product allows for flexibility in adding flavor to food items, confectionery products or chewing gum products. For example, delivery of two or more flavors to a single food item is possible by using encapsulated products containing different flavors in the food item. The delivery of two or more flavors is also possible in confectionery products and chewing gum products.

Advantages of preparing the inventive encapsulated product in this manner are that no heat and no moisture are needed in this process. Additionally and surprisingly, high concentrations of fluoxetine and mesalamine may be incorporated into the final encapsulated product. Furthermore, the encapsulated product of the present inventive subject matter is small enough that when the confectionery or chewing gum product is chewed, the encapsulated product can pass with the saliva and not be disformed by the teeth of the individual chewing.

As is stated above, an advantage of method of the inventive subject matter is that no heat nor moisture is required for forming the encapsulated product. In addition, the encapsulated product of the present inventive subject matter has a uniform active ingredient content and may be strong enough to withstand mechanical pressure both in the processing of the product, and in the chewing of the product in the mouth so that the fluoxetine are released in the stomach.

The following examples are illustrative of preferred embodiments of the invention and are not to be construed as limiting the invention thereto. All percentages are given in weight percent, unless otherwise noted and equal a total of 100%.

### EXAMPLES

### Example 1

### Preparation of 90-mg Fluoxetine HCl Encapsulated Product

65.00% fluoxetine HCl was mixed with 0.005% hydroxypropyl methyl cellulose (HPMC K) 100M, 10.00% hydroxypropylmethyl cellulose (HPMC K) 4M, 3.00% croscarmellose sodium and 15.00% lactose in a shear mixer. After passing the mixture through a no. 30 mesh sieve, the mixture was granulated using 6.00% polyvinylpyrrolidone USP dissolved in 80 ml of isopropyl alcohol. The granulated mixture was passed through a no. 8 mesh and allowed to air dry. When the granulated mixture was dry, it was then passed through a no. 16 mesh. The mixture was next lubricated with a mixture of 1.00% magnesium stearate and 0.50% talc. The final mixture was mixed for 3 minutes. The mixture was loaded into a tableting machine.

A series of caplets was produced using 20 KN of force. The caplets were then enteric coated with cellulose acetate phthalate using conventional coating techniques. In particular, 90.00% (by weight of the enteric coating) acetone was mixed with 1.00% triacetin (a plasticizer), followed by the addition of 9.00% cellulose acetate phthalate. The caplets were coated using a coating pan and spray gun.

Eight (8) uncoated caplets were then tested in vitro for dissolution characteristics. The caplets were loaded into a no. 2 apparatus (paddle, USP) with 1000 ml of pH 6.8 phosphate buffer. The caplets were rotated at 75 rpm (revolutions per minute) and the amount dissolved was determined at 30, 60, 90 and 120 minutes.

The results of the dissolution test are provided Table 1.

### EXAMPLE 2

### Preparation of 90-mg Fluoxetine HCl Encapsulated Product

65.00% fluoxetine HCl was mixed with 15.00% sucrose, 10.00% hydroxypropylmethyl cellulose (HPMC K) 4M, and 3.00% croscarmellose sodium in a shear mixer. After passing the mixture through a no. 30 mesh sieve, the mixture was granulated using 6.00% polyvinylpyrrolidone USP dissolved in 80 ml of isopropyl alcohol. The granulated mixture was passed through a no. 8 mesh and allowed to air dry. When the granulated mixture was dry, it was then passed through a no. 16 mesh. The mixture was next lubricated with a mixture of 1.00% magnesium stearate and 0.50% talc. The final mixture was mixed for 3 minutes. The mixture was loaded into a tableting machine.

A series of capiets was produced using 20 KN of force. The caplets were then enteric coated with cellulose acetate phthalate using conventional coating techniques. In particular, 90.00% (by weight of the enteric coating) acetone was mixed with 1.00% triacetin (a plasticizer), followed by the addition of 9.00% cellulose acetate phthalate. The caplets were coated using a coating pan and spray gun.

Eight (8) uncoated caplets were then tested in vitro for dissolution characteristics. The caplets were loaded into a no. 2 apparatus (paddle, USP) with 1000 ml of pH 6.8 phosphate buffer. The caplets were rotated at 75 rpm (revolutions per minute) and the amount dissolved was determined at 30, 60, 90 and 120 minutes.

The results of the dissolution test are provided Table 1.

### EXAMPLE 3

### Preparation of 90-mg Fluoxetine HCl Encapsulated Product

65.00% fluoxetine HCl was mixed with 10.00% hydroxypropyl methyl cellulose (HPMC K) 100M, 4.00% hydroxypropylmethyl cellulose (HPMC K) 4M, 3.00% croscarmellose sodium and 15.00% lactose in a shear mixer. After passing the mixture through a no. 30 mesh sieve, the mixture was granulated using 6.00% polyvinylpyrrolidone USP dissolved in 80 ml of isopropyl alcohol. The granulated mixture was passed through a no. 8 mesh and allowed to air dry. When the granulated mixture was dry, it was then passed through a no. 16 mesh. The mixture was next lubricated with a mixture of 1.00% magnesium stearate and 0.50% talc. The final mixture was mixed for 3 minutes. The mixture was loaded into a tableting machine.

A series of caplets was produced using 20 KN of force. The caplets were then enteric coated with cellulose acetate phthalate using conventional coating techniques. In particular, 90.00% (by weight of the enteric coating) acetone was mixed with 1.00% triacetin (a plasticizer), followed by the addition of 9.00% cellulose acetate phthalate. The caplets were coated using a coating pan and spray gun.

Eight (8) uncoated caplets were then tested in vitro for dissolution characteristics. The caplets were loaded into a no. 2 apparatus (paddle, USP) with 1000 ml of pH 6.8 phosphate buffer. The caplets were rotated at 75 rpm (revolutions per minute) and the amount dissolved was determined at 30, 60, 90 and 120 minutes.

The results of the dissolution test are provided Table 1.

### EXAMPLE 4

### Preparation of 90-mg Fluoxetine HCl Encapsulated Product

65.00% fluoxetine HCl was mixed with 15.00% sucrose, 10.00% hydroxypropylmethyl cellulose (HPMC K) 100M, and 3.00% croscarmellose sodium in a shear mixer. After passing the mixture through a no. 30 mesh sieve, the mixture was granulated using 6.00% polyvinylpyrrolidone USP dissolved in 80 ml of isopropyl alcohol. The granulated mixture was passed through a no. 8 mesh and allowed to air dry. When the granulated mixture was dry, it was then passed through a no. 16 mesh. The mixture was next lubricated with a mixture of 1.00% magnesium stearate and 0.50% talc. The final mixture was mixed for 3 minutes. The mixture was loaded into a tableting machine.

A series of caplets was produced using 20 KN of force. The caplets were then enteric coated with cellulose acetate phthalate using conventional coating techniques. In particular, 90.00% (by weight of the enteric coating) acetone was mixed with 1.00% triacetin (a plasticizer), followed by the addition of 9.00% cellulose acetate phthalate. The caplets were coated using a coating pan and spray gun.

Eight (8) uncoated caplets were then tested in vitro for dissolution characteristics. The caplets were loaded into a no. 2 apparatus (paddle, USP) with 1000 ml of pH 6.8 phosphate buffer. The caplets were rotated at 75 rpm (revolutions per minute) and the amount dissolved was determined at 30, 60, 90 and 120 minutes.

The results of the dissolution test are provided Table 1.

### EXAMPLE 5

### Preparation of 90-mg Fluoxetine HCl Encapsulated Product

60.00% fluoxetine HCl was mixed with 10.00% hydroxypropyl methyl cellulose (HPMC K) 15M, 4.00% hydroxypropylmethyl cellulose (HPMC K) 4M, 3.00% croscarmellose sodium and 15.00% lactose in a shear mixer. After passing the mixture through a no. 30 mesh sieve, the mixture was granulated using 6.00% polyvinylpyrrolidone USP dissolved in 80 ml of isopropyl alcohol. The granulated mixture was passed through a no. 8 mesh and allowed to air dry. When the granulated mixture was dry, it was then passed through a no. 16 mesh. The mixture was next lubricated with a mixture of 1.00% magnesium stearate and 0.50% talc. The final mixture was mixed for 3 minutes. The mixture was loaded into a tableting machine.

A series of caplets was produced using 20 KN of force. The caplets were then enteric coated with cellulose acetate phthalate using conventional coating techniques. In particular, 90.00% (by weight of the enteric coating) acetone was mixed with 1.00% triacetin (a plasticizer), followed by the addition of 9.00% cellulose acetate phthalate. The caplets were coated using a coating pan and spray gun.

Eight (8) uncoated caplets were then tested in vitro for dissolution characteristics. The caplets were loaded into a no. 2 apparatus (paddle, USP) with 1000 ml of pH 6.8 phosphate buffer. The caplets were rotated at 75 rpm (revolutions per minute) and the amount dissolved was determined at 30, 60, 90 and 120 minutes.

The results of the dissolution test are provided Table 1.

TABLE 1
Dissolution data for 90-mg fluoxetine caplets

| Time | Ex. 1 | Ex. 2 | Ex. 3 | Ex. 4 | Ex. 5 |
|---|---|---|---|---|---|
| 30 Min. | 79.50% | 52.00% | 83.3% | 70.4% | 85.15% |
| 60 Min. | 89.40% | 66.10% | 89.6% | 87.8% | 98.98% |
| 90 Min. | 93.50% | 77.00% | 89.1% | 91.9% | 99.45% |
| 120 Min. | 94.70% | 85.20% | 92.3% | 92.70% | 99.15% |

As can be seen from Table 1, the amount of drug dissolved from the caplets can be controlled by the amount and type of erodible polymer used to within the caplet. The dissolution tests for the product made in Example 5 closely mimic the commercially available 90-mg fluoxetine HCl product.

### EXAMPLE 6

### Preparation of 250-mg Mesalamine Encapsulated Product

73.44% mesalamine was mixed with 22.30% hydroxypropyl methyl cellulose (HPMC K) lOOM in a shear mixer. After passing the mixture through a no. 30 mesh sieve, the mixture was granulated using 3.91% ethyl cellulose N20 dissolved in isopropyl alcohol. The granulated mixture was passed through a no. 8 mesh and allowed to air dry. When the granulated mixture was dry, it was then passed through a no. 16 mesh. The mixture was next lubricated with of 1.00% magnesium stearate. The final mixture was mixed for 3 minutes. The mixture was loaded into a tableting machine.

A series of caplets was produced using 20 KN of force. Fifteen (15) uncoated caplets were then tested in vitro for dissolution characteristics. The caplets were loaded into a no. 2 apparatus (paddle, USP) with 1000 ml of pH 7.5 phosphate buffer. The caplets were rotated at 75 rpm (revolutions per minute) and the amount dissolved was determined at 30, 60, 90, 120, 180, 240, and 300 minutes. The results are provided in Table 2.

### EXAMPLE 7

### Preparation of 250-mg Mesalamine Encapsulated Product

65.00% mesalamine was mixed with 22.30% hydroxypropyl methyl cellulose (HPMC K) 100M in a shear mixer. After passing the mixture through a no. 30 mesh sieve, the mixture was granulated using 3.91% ethyl cellulose N100 dissolved in isopropyl alcohol. The granulated mixture was passed through a no. 8 mesh and allowed to air dry. When the granulated mixture was dry, it was then passed through a no. 16 mesh. The mixture was next lubricated with of 1.00% magnesium stearate. The final mixture was mixed for 3 minutes. The mixture was loaded into a tableting machine.

A series of caplets was produced using 20 KN of force. Fifteen (15) uncoated caplets were then tested in vitro for dissolution characteristics. The caplets were loaded into a no. 2 apparatus (paddle, USP) with 1000 ml of pH 7.5 phosphate buffer. The caplets were rotated at 75 rpm (revolutions per minute) and the amount dissolved was determined at 30, 60, 90, 120, 180, 240, and 300 minutes. The results are provided in Table 2.

### EXAMPLE 8

### Preparation of 250-mg Mesalamine Encapsulated Product

73.44% mesalamine was mixed with 2.77% hydroxypropyl methyl cellulose (HPMC K) 100M and 15.42% hydroxypropyl methyl cellulose (HMPC K) 4M in a shear mixer. After passing the mixture through a no. 30 mesh sieve, the mixture was granulated using 3.97% ethyl cellulose N20 dissolved in isopropyl alcohol. The granulated mixture was passed through a no. 8 mesh and allowed to air dry. When the granulated mixture was dry, it was then passed through a no. 16 mesh. The mixture was next lubricated with of 1.00% magnesium stearate. The final mixture was mixed for 3 minutes. The mixture was loaded into a tableting machine.

A series of caplets was produced using 20 KN of force. Fifteen (15) uncoated caplets were then tested in vitro for dissolution characteristics. The caplets were loaded into a no. 2 apparatus (paddle, USP) with 1000 ml of pH 7.5 phosphate buffer. The caplets were rotated at 75 rpm (revolutions per minute) and the amount dissolved was determined at 30, 60, 90, 120, 180, 240, 300, 360, 420 and 480 minutes. The results are provided in Table 2.

TABLE 2
Dissolution data for 250-mg mesalamine caplets

| Time | Example 6 | Example 7 | Example 8 |
|---|---|---|---|
| 30 Min. | 79.50% | 52.00% | 10.30% |
| 60 Min. | 89.40% | 66.10% | 18.00% |
| 90 Min. | 93.50% | 77.00% | 25.00% |
| 120 Min. | 94.70% | 85.20% | 33.20% |
| 180 Min. | 95.20% | 92.90% | 49.40% |
| 240 Min. | 94.20% | 97.00% | 65.20% |
| 300 Min. | 94.80% | 97.40% | 80.30% |
| 360 Min. | N/A | N/A | 85.50% |
| 420 Min. | N/A | N/A | 94.60% |
| 480 Min. | N/A | N/A | 100.5% |

As can be seen from Table 2, the amount of drug dissolved from the caplets can be controlled by the amount and type of erodible polymer used to within the caplet. The dissolution tests for the product made in Example 8 closely mimic the commercially available 250-mg mesalamine sustained release product.

### Example 9

### Dissolution of 250-mg Mesalamine Product at Various Rotational Speeds

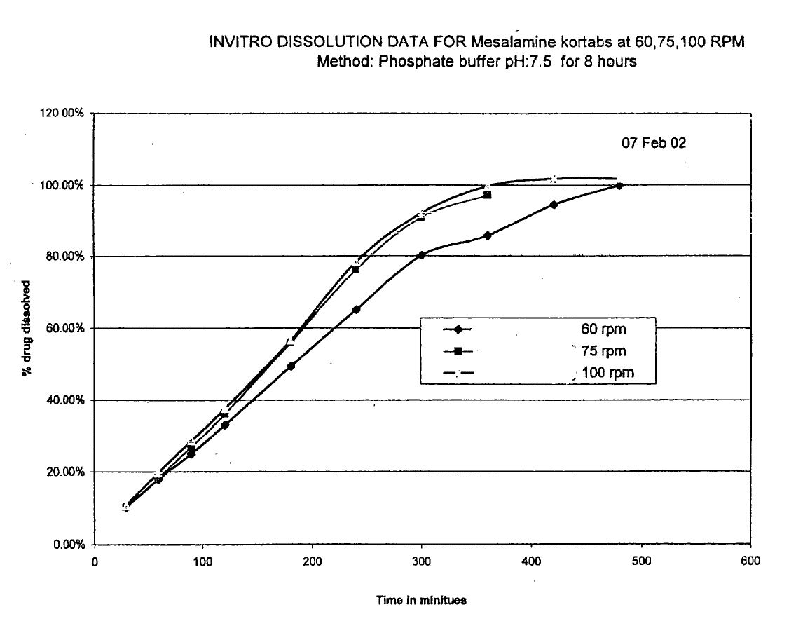

Fifteen (15) caplets of the product prepared in Example 8 were tested in vitro for dissolution rates at different speeds. The caplets were loaded into a no. 2 apparatus (paddle, USP) with 1000 ml of pH 7.5 phosphate buffer. The caplets were rotated at 60 rpm (revolutions per minute), 75 rpm and 100 rpm and the amount dissolved was determined at 30, 60, 90, 120, 180, 240, 300, 360, 420 and 480 minutes. The results are provided in FIG. 1

As can be seen in FIG. 1, the dissolution rate of the mesalamine from the caplets is relatively constant at the different rotational speeds.

The inventive subject matter being thus described, it will be obvious that the same may be varied in many ways. Such variations are not to be regarded as a departure from the spirit and scope of the inventive subject matter, and all such modifications are intended to be included within the scope of the following claims.

1. An encapsulated product having extended or controlled release of fluoxetine, comprising:
a) a pharmaceutically effective amount of fluoxetine HCl;
b) at least one pharmaceutically acceptable excipient;
c) at least one erodible polymer; and
d) at least one lubricating material; and
e) wherein said product is in the form of a caplet having a diameter from about 1 millimeter to about 7 millimeters and a length from about 1 millimeter to about 7 millimeters.

2. The extended or controlled release encapsulated product of claim 1 wherein said erodible polymer is a water soluble polymer.

3. The extended or controlled release encapsulated product of claim 2 wherein said water soluble polymer is selected from the group consisting of sodium carboxymethyl cellulose, hydroxyethyl cellulose, hydroxypropyl cellulose, propylene glycol alginate, sodium alginate, carboxymethyl cellulose, and mixtures thereof.

4. The extended or controlled release encapsulated product of claim 1 wherein said erodible polymer is a water insoluble polymer.

5. The extended or controlled release encapsulated product of claim 4 wherein said water insoluble polymer is selected from the group consisting of cellulose acetate, ethyl cellulose, cellulose acetate methyl carbamate, methylcarbamate, polydiethylaminomethylstyrene, ethyl cellulose, cellulose acetate, cellulose diacetate, cellulose triacetate, cellulose alkanylate, monoalkenytes, dialkenytes, trialkenytes, mono-, di- and tri-arolyates, cellulose trivalerate, cellulose trioctanoate, cellulose tripionate, celluslose diesters, cellulose disuccinate, cellulose acetate valerate, cellulose acetaldehyde, dimethylcellulose acetate, cellulose dimethylaminoacetate, semipermeable sulfonated polystyrenes, semipermeable styrenes, hydroxypropylmethyl celluose and mixtures thereof.

6. The extended or controlled release encapsulated product of claim 1 wherein said lubricating material is selected from the group consisting of: fats, emulsifiers, waxes, magnesium stearate, calcium stearate, talc, starches, silicon dioxide, and mixtures thereof.

7. The extended or controlled release encapsulated product of claim 1 wherein said diameter is about 3 millimeters and said length is about 3 millimeters.

8. The extended or controlled release encapsulated product of claim 1 wherein the product is coated with an enteric coating.

9. The extended or controlled release encapsulated product of claim 8 wherein the enteric coating is cellulose acetate phthalate.

10. The extended or controlled release product of claim 1, wherein said excipients comprise povolone and lactose.

11. A pulsating release encapsulated product for use with fluoxetine HCl, comprising:
a) a pharmaceutically effective amount of fluoxetine HCl;
b) at least one pharmaceutically acceptable excipient;
c) at least two erodible polymers, each of said erodible polymers having a different rate of dissolution or dissolving at a different pH; and
d) at least one lubricating material; and
e) wherein said product is in the form of a caplet having a diameter from about 1 millimeter to about 7 millimeters and a length from about 1 millimeter to about 7 millimeters.

12. The pulsating release encapsulated product of claim 11 wherein said erodible polymer is a water soluble polymer.

13. The pulsating release encapsulated product of claim 12 wherein said water soluble polymer is selected from the group consisting of sodium carboxymethyl cellulose, hydroxyethyl cellulose, hydroxypropyl cellulose, propylene glycol alginate, sodium alginate, carboxymethyl cellulose and mixtures thereof.

14. The pulsating release encapsulated product of claim 11 wherein said erodible polymer is a water insoluble polymer.

15. The pulsating release encapsulated product of claim 14 wherein said water insoluble polymer is selected from the group consisting of cellulose acetate, ethyl cellulose, cellulose acetate methyl carbamate, methylcarbamate, polydiethylaminomethylstyrene, ethyl cellulose, cellulose acetate, cellulose diacetate, cellulose triacetate, cellulose alkanylate, monoalkenytes, dialkenytes, trialkenytes, mono-, di- and tri-arolyates, cellulose trivalerate, cellulose trioctanoate, cellulose tripionate, celluslose diesters, cellulose disuccinate, cellulose acetate valerate, cellulose acetaldehyde, dimethylcellulose acetate, cellulose dimethylaminoacetate, semipermeable sulfonated polystyrenes, semipermeable styrenes, hydroxypropylmethyl cellulose and mixtures thereof.

16. The pulsating release encapsulated product of claim 11 wherein said lubricating material is selected from the group consisting of: fats, emulsifiers, waxes, magnesium stearate, calcium stearate, talc, starches, silicon dioxide, and mixtures thereof.

17. The pulsating release encapsulated product of claim 11 wherein said diameter is about 3 millimeters and said length is about 3 millimeters.

18. The pulsating release encapsulated product of claim 11 wherein the product is coated with an enteric coating.

19. The pulsating release encapsulated product of claim 11 wherein the enteric coating is cellulose acetate phthalate.

20. An encapsulated product having extended or controlled release of fluoxetine, comprising:
a) a pharmaceutically effective amount of mesalamine;
b) at least one pharmaceutically acceptable excipient;
c) at least one erodible polymer; and
d) at least one lubricating material; and
e) wherein said product is in the form of a caplet having a diameter from about 1 millimeter to about 7 millimeters and a length from about 1 millimeter to about 7 millimeters.

21. A pulsating release encapsulated product for use with fluoxetine HCl, comprising a capsule having a plurality of caplets, said caplets comprising:
a) a pharmaceutically effective amount of fluoxetine HCl;
b) at least one pharmaceutically acceptable excipient;
c) at least one erodible polymer;
d) at least one lubricating material; and
e) wherein said caplet has a diameter from about 1 millimeter to about 7 millimeters and a length from about 1 millimeter to about 7 millimeters; and
wherein at least one of said plurality of caplets is prepared from an erodible polymer having a first dissolution rate, and at least another of said plurality of caplets is prepared from another erodible polymer having a second dissolution rate, and said first dissolution rate is not equal to said second dissolution rate.

22. The pulsating release encapsulated product of claim 21, wherein said erodible polymer is a water soluble polymer.

23. The pulsating release encapsulated product of claim 22, wherein said water soluble polymer is selected from the group consisting of sodium carboxymethyl cellulose, hydroxyethyl cellulose, hydroxypropyl cellulose, propylene glycol alginate, sodium alginate, carboxymethyl cellulose and mixtures thereof.

24. The pulsating release encapsulated product of claim 21, wherein said erodible polymer is a water insoluble polymer.

25. The pulsating release encapsulated product of claim 24, wherein said water insoluble polymer is selected from the group consisting of cellulose acetate, ethyl cellulose, cellulose acetate methyl carbamate, methylcarbamate, polydiethylaminomethylstyrene, ethyl cellulose, cellulose acetate, cellulose diacetate, cellulose triacetate, cellulose alkanylate, monoalkenytes, dialkenytes, trialkenytes, mono-, di- and tri-arolyates, cellulose trivalerate, cellulose trioctanoate, cellulose tripionate, celluslose diesters, cellulose disuccinate, cellulose acetate valerate, cellulose acetaldehyde, dimethylcellulose acetate, cellulose dimethylaminoacetate, semipermeable sulfonated polystyrenes, semipermeable styrenes, hydroxypropylmethyl cellulose and mixtures thereof.

26. The pulsating release encapsulated product of claim 21, wherein said lubricating material is selected from the group consisting of: fats, emulsifiers, waxes, magnesium stearate, calcium stearate, talc, starches, silicon dioxide, and mixtures thereof.

27. The pulsating release encapsulated product of claim 21, wherein said diameter is about 3 millimeters and said length is about 3 millimeters.

28. The pulsating release encapsulated product of claim 21, wherein the product is coated with an enteric coating.

29. The pulsating release encapsulated product of claim 21, wherein the enteric coating is cellulose acetate phthlate.

30. A pulsating release encapsulated product for use with mesalamine, comprising:
a) a pharmaceutically effective amount of mesalamine;
b) at least one pharmaceutically acceptable excipient;
c) at least two erodible polymers, each of said erodible polymers having a different rate of dissolution or dissolving at a different pH; and
d) at least one lubricating material; and
e) wherein said product is in the form of a caplet having a diameter from about 1 millimeter to about 7 millimeters and a length from about 1 millimeter to about 7 millimeters.
